(19) United States
(12) Patent Application Publication
(10) Pub. No.: US 2005/0086714 A1
(43) Pub. Date: Apr. 21, 2005

(54) Eucalyptus transformation method

(75) Inventors: Jia-Long Yao, Auckland (NZ); Kui Lin-Wang, Auckland (NZ)

Correspondence Address: SPECKMAN LAW GROUP PLLC, 1501 WESTERN AVE, SEATTLE, WA, 98101, US

(73) Assignee: AGRIGENESIS BIOSCIENCES LIMITED, Auckland (NZ)

(21) Appl. No.: 10/960,848

(22) Filed: Oct. 6, 2004

Related U.S. Application Data
(60) Provisional application No. 60/508,944, filed on Oct. 6, 2003.

(51) Int. Cl. C12N 15/82; A01H 5/00

(52) U.S. Cl. 800/278; 800/323

(57) ABSTRACT

Methods for producing genetically modified plants of the Eucalyptus species particularly Eucalyptus grandis, are provided. The methods involve transformation of internode stem segments with a desired genetic construct using Agrobacterium-mediated techniques and regeneration of the transformed plant material. Preferred culture media, including selection media, and improved plant culture techniques are disclosed.

### REFERENCE TO RELATED APPLICATIONS

This application claims priority to U.S. Provisional Patent Application No. 60/508,944, filed Oct. 6, 2003.

### FIELD OF THE INVENTION

This application relates to a method for transformation and regeneration of commercially important Eucalyptus species. In particular, the inventive method produces high efficiency transformation of stem internode tissues using Agrobacterium with rapid regeneration of shoots.

### BACKGROUND

Eucalyptus is one of the most commercially important hardwoods in the world, with its wood being used to produce pulp and paper, and as an energy source. Eucalyptus is also used in the chemical and medical industries, to provide shade and shelter, and as a source of essential oils. At present, Eucalyptus trees in commercial forestry are propagated using seed or rooted cuttings from superior trees selected using traditional plant breeding techniques. As trees have a long life cycle, traditional breeding of Eucalyptus species is slow and has many limitations. Recent advances in genetic engineering make it possible to stably integrate novel and useful genes into plants using recombinant DNA technology. These recombinant techniques have the potential for providing more rapid improvements of Eucalyptus plant stock than is possible with traditional breeding methods.

As trees are highly heterozygous, propagation from seed and seedling materials results in the loss of some superior characteristics in many trees because of gene segregation in the progeny population. Therefore, for more uniform forestry planting, propagation from rooted cuttings and micropropagation from vegetative tissue of the superior trees are preferred (Leroux and Staden, Tree Physiol. 9:435-477, 1991). Similarly, vegetative tissues (e.g. stem internodes) rather than seedling tissues (e.g. hypocotyls and cotyledons) should preferably be used as the starting material for genetic transformation in order to directly improve a superior tree selected through traditional breeding and avoid the segregation of superior traits.

There are two general strategies for transforming plant cells. One uses Agrobacterium to transfer DNA, known as T-DNA, into plant cells (Zupan et al., Plant J. 23:11-28, 2000). The other strategy involves ‘direct DNA transfer’ into plant cells or protoplasts by means of various techniques, such as microinjection of DNA into plant cells, polyethylene glycol (PEG)-mediated transformation of protoplasts, particle bombardment, and electroporation. These and other transformation techniques have been reviewed by Twyman et al. (in Plant Biotechnology and Transgenic Plants. Marcel-Dekker Inc. NY. pp.111-141, 2002). Of the two general strategies, Agrobacterium-mediated transformation is the most widely used at present, as it is simple, low cost and highly efficient. Compared to direct DNA transfer, Agrobacterium-mediated transformation generally produces transgenic plants with lower transgene copy numbers. The transfer of a single copy transgene is a highly desirable characteristic which reduces transgene silencing.

There are two general classes of transgene silencing: position effect and homology-dependent. In position effect silencing, the flanking plant DNA and/or chromosomal location negatively influences the expression of single transgene loci. The chromatin structure around each transgene locus may differ and may result in variable accessibility to transcription factors (Dean et al., Nucleic Acids Res. 16:9267-9283, 1988). Transgene inactivation in aspen trees has been reported to be frequently associated with the presence of AT-rich flanking plant DNA (Kumar & Fladung, Planta 213:731-740, 2001).

Homology-dependent gene silencing occurs when multiple copies of a transgene are present in a genome. Multiple copies of the same gene construct may be present at different loci or at one locus in a genome. When they are located at one locus, they are arranged as direct or inverted repeat structures. The repeat transgene structure is often the target for gene silencing.

The structure and complexity of the transgene locus may depend on the particular Agrobacterium strain used for transformation. T-DNA is organized predominantly in inverted repeat structures in plants transformed with Agrobacterium tumefaciens C58 derivatives (Jorgensen et al., Mol. Gen. Genet. 207:471-477, 1987). The Agrobacterium strains EHA101, EHA105 and AGL1 are all C58 derivatives and are hypervirulent strains. Complete removal of T-DNA from the resident Ti plasmid in these strains is unconfirmed (Hood et al., J Bacteriol. 168:1291-1301, 1986). These strains are often chosen for transforming recalcitrant species as they may give a higher level of transformation. However, this high level of transformation may be gained at the cost of stable transgene expression. By contrast, LBA4404 is a less-virulent strain of Agrobacterium and all T-DNA in the residential Ti plasmid pAL4404 is eliminated (Hoekema et al., Nature 303:179-180, 1983). LBA4404 has been successfully used to transform many species and gives more reliable transgene expression.

Transgene silencing is undesirable since it reduces the efficiency and reliability of transgene expression. Single copy lines are therefore preferred. Stable expression of transgene(s) is important for commercial use of genetic transformation in long-lived tree species.

A complete plant transformation protocol includes not only transforming cells with foreign DNA, but also regenerating whole plants from the transformed cells. Therefore, a suitable plant regeneration system is a prerequisite for developing a transformation protocol. Depending on the species, different regeneration systems have been used for transformation, including somatic embryogenesis and organogenesis systems. Because of the difficulty of developing a somatic embryogenesis system for most species, only a small number of plant species are capable of being regenerated in this way. A commercially reliable organogenesis system should have the following features: (1) regeneration from single cells to avoid production of chimeric transgenic plants; (2) the regenerable cells should also be transformable; and (3) regeneration should occur directly from the originally transformed cells without a callus induction phase in order to avoid somaclonal variation and reduce the time interval between transformation and regeneration, thereby allowing transgenic plants to maintain all the superior characteristics and to be produced rapidly.

Several protocols have previously been described in the scientific and patent literature for Eucalyptus transformation. Most protocols use explants derived from embryos (Serrano et al., J Exp. Bot. 47:285-290, 1996), young seedlings (Moralejo et al., Plant Cell Rep. 16:299-303, 1997), or cotyledons and hypocotyls (Ho et al., Plant Cell Rep. 17:675-680, 1998; Kawazu et al. Tree improvement for sustainable tropical forestry. QFRI-IUFRO Conference, Queensland, Australia, 2:492-497, 1996). Other protocols are useful for transforming the model species E. camaldulensis (Mullens et al., Plant Cell Rep. 16: 787-791, 1997), but are not satisfactory for commercially important forestry species, such as E. grandis. Some protocols require a particular cytokine additive to promote regeneration, with regeneration involving a callus induction phase (International Patent Publication WO 96/25504). Other protocols involve transformation of nodal stem tissues with poorly transformable meristem cells which give rise to chimeric transgenic shoots (US published patent application US 2002-0016981 A1).

Previously described protocols use wild type Agrobacterium (Luciana et al., Plant Cell Rep. 16:299-303, 1997) or hypervirulent strains (for example, EHA101, EHA105, AGL1, GV3850) of Agrobacterium (International Patent Publication WO 96/25504; Mullins et al., Plant Cell Rep. 16:787-791, 1997; International Patent Publication WO 97/25434; Ho et al., Plant Cell Rep. 17:675-680, 1998; US published patent application US 2002-0016981 A1; US published patent application US 2003-0033639 A1). Transgenic plants produced using wild type Agrobacterium strain are often abnormal physiologically and morphologically due to the over-production of hormones from the genes carried by the wild type Ti plasmid. Transformation with hypervirulent strains is likely to produce plants with multiple T-DNA insertions and complex transgene loci as discussed above.

### SUMMARY OF THE INVENTION

The present invention provides rapid and efficient methods for transforming and regenerating commercially useful Eucalyptus species that may be effectively used in commercial forestry. The inventive methods involve transforming internode stem segments of Eucalyptus species, such as E. grandis, with a non-hypervirulent Agrobacterium strain containing a DNA sequence, or polynucleotide, of interest and regenerating transgenic shoots from the transformed internode segments. Such methods provide more single copy T-DNA integration than is obtained using hypervirulent strains of Agrobacterium. The present invention thus provides a rapid regeneration system which avoids somaclonal variation and reduces the time required for transgenic plant production. Furthermore, the use of stem internode segments advantageously provides a high percentage of transformable, regenerable cells which are capable of stable transgene expression.

More specifically, one or more genetic construct(s) comprising a reporter gene, preferably the kanamycin resistance gene, and the genetic material desired to be introduced is transformed into a non-hypervirulent Agrobacterium strain, preferably the strain LBA4404. The target plant material from the Eucalyptus species is then inoculated with Agrobacterium carrying the genetic construct of interest.

Preferred tissue explants of the target plant comprise internode stem segments collected from in vitro grown shoot cultures, preferably cultured on EMA4 medium (see Table 2 below). The internode segments are placed in co-cultivation medium, preferably medium EuCo19 (see Table 2) and preferably in a horizontal orientation, and are incubated with the transformed Agrobacterium culture to inoculate the internode segments with the desired genetic material. Following inoculation, regeneration of shoots from the Agrobacterium infected internode segments is promoted in tissue culture using a combination of media containing kanamycin. In a preferred embodiment, the transformed internode segments are grown on medium EuSe7 (see Table 2 below) containing 30 mg/l kanamycin for 4 weeks, then transferred to medium EuSe7 containing 50 mg/l kanamycin for 4 weeks. The resulting kanamycin-resistant shoots are then transferred to medium EuRT3, which contains 250 mg/l timentin and 50 mg/l kanamycin (see Table 2 below), for four weeks for shoot elongation.

Following regeneration of transformed shoots, the shoots are transferred to a rooting medium and roots are generated using techniques that are well known in the art. Rooted plants are then transferred into soil to complete the transformation and regeneration procedure. The plants, which include the genetic material introduced using the genetic construct, may then be grown to maturity to provide genetically modified mature plants. Materials obtained from the mature plants, such as timber, wood pulp, fuel wood and the like, also contain the genetic modification.

The transformation and regeneration methods of the present invention can be employed to provide plants having all the superior characteristics provided by the DNA of interest, quickly and in a reproducible, efficient and low cost manner. The inventive methods are suitable for commercial production of genetically modified Eucalyptus species, including commercially important forestry species such as Eucalyptus grandis.

These methods may be employed to introduce new genes, additional copies of existent genes, or non-coding portions of a genome, into selected clones with little disturbance of the plant's genome. Genetic material may be introduced that produces desirable traits, such as insect tolerance, disease resistance, herbicide tolerance, male sterility, rooting ability, cold tolerance, drought tolerance, salinity tolerance, and modification of wood properties and growth rates, and the like. The genetic material introduced may be homologous or heterologous to the genome of the target plant.

The present invention also contemplates plants, plant materials, and plant products derived from genetically modified plants produced according to the methods of the present invention. The term “plants” includes mature and immature plants grown from plantlets produced according to methods of the present invention, as well as progeny of such plants and plants propagated using materials from such plants. The term “plant materials” includes plant cells or tissues such as seeds, flowers, bark, stems, etc. of all such plants. The term “plant products” includes any materials derived from plant materials, such as wood products, pulp products, and the like.

### BRIEF DESCRIPTION OF THE DRAWINGS

FIG. 1 shows shoot regeneration on internode segments of different ages collected from a shoot continuously sub-cultured on the EEM medium or in its first sub-culture on the EMA4 medium.

FIG. 2 shows shoot regeneration on internode segments of different ages collected from a shoot continuously sub-cultured on the EMA4 medium.

FIG. 3 shows a high frequency of shoot regeneration from young internode segments grown on EuCo14 medium.

FIG. 4 shows strong transient GUS expression on internode explants after inoculation with Agrobacterium LBA4404 containing the binary vector pART69.

FIG. 5 shows regeneration of shoots from transformed internode tissues on kanamycin.

FIG. 6 shows elongation and rooting of a transgenic line in the EuRt3 medium containing 50 mg/l kanamycin at 1 week (FIG. 6A) and at 4 weeks (FIG. 6B).

FIG. 7 shows stable GUS expression in leaves isolated from kanamycin-resistant transgenic plants. The leaf taken from non-transgenic control (on the left) is GUS negative and the leaf taken from the transgenic plant produced using the internode system (GIN001; on the right) is GUS positive.

FIG. 8 shows DNA fragments PCR amplified from Eucalyptus plants transformed with the pART69 vector. The PCR primers were designed to amplify an 804 bp fragment from the nptII gene and a 677 bp fragment from the GUS gene. PCR template DNA samples were from pART69 plasmid (1), a non-transgenic (2) and 6 independent transgenic plants (1-8). nptII and GUS gene fragments were amplified from pART69 positive control (1) and 6 transgenic plants (3-8) but not from the non-transgenic negative control plant (2). M is the 1 kb Plus DNA Ladder (Gibco BRL, Carlsbad, Calif.).

### DETAILED DESCRIPTION

Using the methods and materials of the present invention, the genome of a target plant, such as a Eucalyptus species, may be modified by incorporating homologous or heterologous genetic material. Additional copies of genes encoding certain polypeptides, or functional portions of certain polypeptides, such as enzymes involved in a biosynthetic pathway, may be introduced into a target plant using the methods of the present invention to increase the level of a polypeptide of interest. Similarly, a change in the level of a polypeptide of interest in the target plant may be achieved by transforming the target plant with antisense copies of genes encoding the polypeptide of interest, or a functional portion of the polypeptide of interest. Additionally, the number of copies of genes encoding different polypeptides, such as enzymes in a biosynthetic pathway, may be manipulated to modify the relative amount of each polypeptide synthesized, leading to the formation of an end product having a modified composition. Non-coding portions of polynucleotides, such as regulatory polynucleotides and polynucleotides encoding regulatory factors, such as transcription factors, and/or finctional portions of transcription factors, and/or antisense copies of such regulatory factors, may also be introduced to target plant material to modulate the expression of certain polypeptides. These materials are exemplary of the types of genetic material suitable for modifying the genome of the target plant material. Numerous other materials may also be introduced.

The methods of the present invention preferably employ shoot cultures of the target plant material as a starting material. Such shoot cultures are preferably grown in vitro from seeds grown on ½ strength MS (Murashige & Skoog) medium (Sigrna, St Louis Mo.) or from vegetative tissues of superior mature trees. A method for establishing shoot cultures from vegetative tissues of mature trees bas been described by Sharma and Ramamurthy (Plant Cell Rep. 19:511-518, 2000). Approximately four weeks after germination, the resulting shoot cultures are transferred to a multiplication and elongation medium. Preferably, the multiplication and elongation medium comprises full strength MS medium, sucrose, benzylaminopurine (BA) and naphthalene acetic acid (NAA). Preferred multiplication and elongation media are described in detail in Example 1 below.

The “genetic material” transformed into the target plant material includes one or more genetic construct(s) comprising one or more polynucleotide(s) desired to be introduced into the target plant material, and a reporter construct. Genetic constructs introduced into the target plant material may comprise, genetic material that is homologous and/or heterologous to the target plant material, and may include polynucleotides encoding a polypeptide or a functional portion of a polypeptide, polynucleotides encoding a regulatory factor, such as a transcription factor, non-coding polynucleotides such as regulatory polynucleotides, and antisense polynucleotides that inhibit expression of a specified polypeptide. The genetic construct may additionally comprise one or more regulatory elements, such as one or more promoters. The genetic construct is preferably functional in the target plant.

According to one embodiment, the genetic constructs used in connection with the present invention include an open reading frame coding for at least a functional portion of a polypeptide of interest in the target plant material. A polypeptide of interest may be a structural or functional polypeptide, or a regulatory polypeptide such as a transcription factor. As used herein, the “functional portion” of a polypeptide is that portion which contains the active site essential for affecting the metabolic step, i.e. the portion of the molecule that is capable of binding one or more reactants or is capable of improving or regulating the rate of reaction. The active site may be made up of separate portions present on one or more polypeptide chains and will generally exhibit high substrate specificity.

A target plant may be transformed with more than one genetic construct, thereby modulating a biosynthetic pathway for the activity of more than one polypeptide, affecting an activity in more than one tissue or affecting an activity at more than one expression time. Similarly, a genetic construct may be assembled containing more than one open reading frame coding for a polypeptide or more than one non-coding region of a gene.

The word “polynucleotide(s),” as used herein, means a polymeric collection of nucleotides and includes DNA and corresponding RNA molecules, both sense and anti-sense strands, and comprehends cDNA, genomic DNA and recombinant DNA, as well as wholly or partially synthesized polynucleotides. A polynucleotide may be an entire gene, or any portion thereof. Operable anti-sense polynucleotides may comprise a fragment of the corresponding polynucleotide, and the definition of “polynucleotide” includes all such operable anti-sense fragments. A “polynucleotide of interest”, as used herein, is a polynucleotide that is homologous or heterologous to the genome of the target plant and alters the genome of the target plant.

As used herein, the term “polypeptide” encompasses amino acid chains of any length, including full length proteins, wherein amino acid residues are linked by covalent peptide bonds.

When the genetic construct comprises a coding portion of a polynucleotide, the genetic construct further comprises a gene promoter sequence and a gene termination sequence operably linked to the polynucleotide to be transcribed. The gene promoter sequence is generally positioned at the 5′ end of the polynucleotide to be transcribed, and is employed to initiate transcription of the polynucleotide. Promoter sequences are generally found in the 5′ non-coding region of a gene but they may exist in introns or in the coding region. When the construct includes an open reading frame in a sense orientation, the gene promoter sequence also initiates translation of the open reading frame. For genetic constructs comprising either an open reading frame in an antisense orientation or a non-coding region, the gene promoter sequence may comprise a transcription initiation site having an RNA polymerase binding site.

A variety of gene promoter sequences which may be usefully employed in the genetic constructs of the present invention are well known in the art. The promoter gene sequence, and also the gene termination sequence, may be endogenous to the target plant host or may be exogenous, provided the promoter is functional in the target host. For example, the promoter and termination sequences may be from other plant species, plant viruses, bacterial plasmids and the like.

Factors influencing the choice of promoter include the desired tissue specificity of the construct, and the timing of transcription and translation. For example, constitutive promoters, such as the 35S Cauliflower Mosaic Virus (CaMV 35S) promoter, will affect the activity of a polypeptide in all parts of the plant. Use of a tissue specific promoter will result in production of the desired sense or antisense RNA only in the tissue of interest. With genetic constructs employing inducible gene promoter sequences, the rate of RNA polymerase binding and initiation may be modulated by external stimuli, such as light, heat, anaerobic stress, alteration in nutrient conditions and the like. Temporally regulated promoters may be employed to effect modulation of the rate of RNA polymerase binding and initiation at a specific time during development of a transformed cell. Preferably, the original promoters from the enzyme gene in question, or promoters from a specific tissue-targeted gene in the organism to be transformed, such as Eucalyptus, are used. Other examples of gene promoters which may be usefully employed in the present invention include mannopine synthase (mas), octopine synthase (ocs) and those reviewed by Chua et al., (Science 244:174-181, 1989). Multiple copies of promoters, or multiple promoters, may be used to selectively stimulate expression of a polynucleotide comprising a part of the genetic construct.

The gene termination sequence, which is located 3′ to the DNA sequence to be transcribed, may come from the same gene as the gene promoter sequence or may be from a different gene. Many gene termination sequences known in the art may be usefully employed in the present invention, such as the 3′ end of the Agrobacterium tumefaciens nopaline synthase gene. However, preferred gene terminator sequences are those from the original polypeptide gene, or from the target species being transformed.

The genetic constructs of the present invention also comprise a reporter gene and/or a selection marker that is effective in target plant cells to permit the detection of transformed cells containing the genetic construct. Such reporter genes and selection markers, which are well known in the art, typically confer resistance to one or more toxins. A chimeric gene that expresses β-D-glucuronidase (GUS) in transformed plant tissues but not in bacterial cells is a preferred selection marker for use in methods of the present invention. Plant material expressing GUS is resistant to antibiotics such as kanamycin. Another suitable marker is the nptII gene, whose expression results in resistance to kanamycin or hygromycin, antibiotics which are generally toxic to plant cells at a moderate concentration (Rogers et al. in Weissbach A and Weissbach H, eds., Methods for Plant Molecular Biology, Academic Press Inc., San Diego, Calif., 1988). Alternatively, the presence of the desired construct in transformed cells may be determined by means of other techniques that are well known in the art, such as Southern and Western blots.

Techniques for operatively linking the components of the genetic constructs used to transform target plant materials are well known in the art and include the use of synthetic linkers containing one or more restriction endonuclease sites as described, for example, by Sambrook et al., (Molecular cloning: a laboratory manual, CSHL Press: Cold Spring Harbor, N.Y., 1989). Genetic constructs used in the inventive methods may be linked to a vector having at least one replication system, for example, E. coli, whereby after each manipulation, the resulting construct can be cloned and sequenced and the correctness of the manipulation determined.

For applications where amplification of a polypeptide is desired, an open reading frame encoding the polypeptide of interest, or a polynucleotide encoding a regulatory factor that modulates expression of the polypeptide of interest, may be inserted in the genetic construct in a sense orientation, such that transformation of a target plant with the genetic construct will produce an increase in the number of copies of the gene or an increase in the expression of the gene and, consequently, an increase in the amount of the polypeptide. When down-regulation of a polypeptide is desired, an open reading frame encoding the polypeptide of interest may be inserted in the genetic construct in an antisense orientation, such that the RNA produced by transcription of the polynucleotide is complementary to the endogenous mRNA sequence. This, in turn, will result in a decrease in the number of copies of the gene and therefore a decrease in the amount of enzyme. Alternatively, modulation may be achieved by inserting a polynucleotide encoding a regulatory element, such as a promoter or a transcription factor, that modulates expression of the polynucleotide encoding the polypeptide of interest.

In another embodiment, the genetic construct used to transform the target plant material may comprise a nucleotide sequence including a non-coding region of a gene coding for a polynucleotide of interest, or a nucleotide sequence complementary to such a non-coding region. As used herein the term “non-coding region” includes both transcribed sequences which are not translated, and non-transcribed sequences within about 2000 base pairs 5′ or 3′ of the translated sequences or open reading frames. Examples of non-coding regions which may be usefully employed in the inventive constructs include introns and 5′-non-coding leader sequences. Transformation of a target plant with such a genetic construct may lead to a reduction in the amount of a selected polypeptide synthesized by the plant by the process of cosuppression, in a manner similar to that discussed, for example, by Napoli et al., (Plant Cell 2:279-290, 1990) and de Carvalho Niebel et al., (Plant Cell 7:347-358, 1995).

Genetic constructs may be used to transform a variety of plants using the methods of the present invention, including monocotyledonous (e.g., grasses, corn, grains, oat, wheat and barley), dicotyledonous (e.g., Arabidopsis, tobacco, legumes, alfalfa, oaks, Eucalyptus, maple), and Gymnosperms (e.g., Scots pine (Aronen, Finnish Forest Res. Papers, Vol. 595, 1996), white spruce (Ellis et al., Biotechnology 11:84-89, 1993), and larch (Huang et al., In vitro Cell 27:201-207, 1991)). In preferred embodiments, the genetic constructs are employed to transform “woody plants,” which are herein defined as a tree or shrub whose stem lives for a number of years and increases in diameter each year by the addition of woody tissue. Preferably, the target plant is preferably selected from the group consisting of Eucalyptus species, more preferably from the group consisting of commercially important Eucalyptus species. The target plant is most preferably Eucalyptus grandis.

Transfer of one or more genetic constructs into target plant shoots is accomplished using Agrobacterium-mediated transformation techniques. Numerous Agrobacterium strains are suitable and are commercially available. Preferably the Agrobacterium strain is a non-hypervirulent strain, such as Agrobacterium tumefaciens strain LBA4404. Methods for transforming a population of the Agrobacterium strain with a genetic construct are well known. A preferred method for transforming the Agrobacterium culture with the genetic construct of interest is described below in Example 3.

Mature shoots of the target plant material prepared as described above are selected for transformation. Shoots of the target plant material are preferably allowed to grow from 8 to 10 days before internode stem segments are collected from the shoots. In a preferred embodiment, the internode segments are taken from close to the top of the shoots. Most preferably, the top two internode segments are transformed to incorporate the desired genetic material. The selected internode stem segments are then inoculated with the Agrobacterium culture prepared as described above.

Inoculation of internode segments with the Agrobacterium suspension takes place under conditions that optimize infection of the segments. Preferably the internode segments are placed in a horizontal orientation on a co-cultivation medium. A preferred co-cultivation medium comprises MS medium with about 20 g/l sucrose, 3 mg/l zeatin, and 0.01 mg/l thiadazuron (TDZ), supplemented with about 100 μM acetosyringone. Agrobacterium cells are then added to each internode explant and co-cultivated, preferably for around three days under a low intensity light at 22° C.

Following the co-cultivation period, the internode segments are cultured in a first selection medium preferably comprises MS medium, sucrose, zeatin, TDZ, timentin and a selection agent, such as kanamycin at a concentration of about 30 mg/l for a period of four weeks and then transferred to the same medium but containing kanamycin at a concentration of about 50 mg/l for an additional four weeks.

Putative transformed shoots are transferred to a shoot elongation medium. A preferred shoot elongation medium comprises half strength MS medium, sucrose at a concentration of about 20 g/l, BA at a concentration of about 0.2 mg/l, NAA at a concentration of about 0.05 mg/l, timentin at a concentration of about 250 mg/l and kanamycin at a concentration of about 50 mg/l. GUS staining of the stem segments of the shoots may also be monitored to eliminate chimeric shoots. This may be accomplished by taking cross sections of the basal regions of putative transformed shoots and staining overnight according to methods described in Stomp, “Histochemical localization of β-glucuronidase,” in GUS Protocols: using the GUS gene as a reporter of gene expression, pp. 103-113, 1992. To ensure chimera-free transgenic plants, only the shoots showing 100% GUS staining may be selected for plantlet development.

Transformed shoots are transferred to a suitable rooting medium. A preferred rooting medium is the same as the shoot elongation medium described above. Rooting is accomplished in a period of from about two to four weeks and may involve an initial culture period in the dark to allow initial root development, followed by transfer to standard photoperiod conditions. During elongation and rooting, explants may be transferred to larger culture vessels, such as Magenta boxes. Rooted shoots, or plantlets, may be transferred to a growth medium, such as soil, and grown to mature, genetically modified plants. Genetically modified plants produced according to the methods disclosed herein may be reproduced, for example, using standard clonal propagation techniques such as axillary bud multiplication techniques.

The following examples are offered by way of illustration and not by way of limitation.

### EXAMPLE 1

### PLANT MATERIALS

In studies using leaf primordia as explants, a large number of Eucalyptus clones were tested for their ability to regenerate and to be transformed using Agrobacterium. A number of superior clones were identified (Table 1) which were used to establish a regeneration and Agrobacterium-mediated transformation system employing internode segments as explant materials.

TABLE 1
Eucalyptus plant materials used in developing internode transformation system

| Clone | | Seedlot/ | | |
|---|---|---|---|---|
| prefix | Species | Cultivar/Batch | Source | Address |
| 8 | grandis | | Trees & Technology | Te Teko, New |
| | | | | Zealand |
| 9 | grandis x | | Same as above | Same as above |
| | nitens | | | |
| A001 | grandis | 24/09/1999 | AustraHort Pty | PO Box Cleveland |
| | | | Limited, Australia | QLD 4163, |
| | | | | Australia |
| B | grandis | 27/10/2000 | Same as above | Same as above |
| C101 | grandis | #1192 | Zimbabwe | |
| D109 | grandis | #17709 | Australian Tree Seed | PO Box E4008, |
| | | | Centre, CSIRO | Kingston ACT |
| | | | Forestry and Forest | 2604, Australia |
| | | | Products | |
| E | grandis | #18342 | Same as above | Same as above |
| F | grandis | #12080 | Same as above | Same as above |
| G | grandis | #16723 | Same as above | Same as above |
| H | grandis | #18146 | Same as above | Same as above |
| I | grandis | #18701 | Same as above | Same as above |
| J | grandis | #18705 | Same as above | Same as above |
| K | grandis | #19313 | Same as above | Same as above |
| L | grandis | #19967 | Same as above | Same as above |
| M | grandis | Rose Gum | New Zealand Tree | PO Box 435, |
| | | | Seeds | Rangiora, North |
| | | | | Canterbury |
| N | gunnii | Cider Gum | Same as above | Same as above |
| O | grandis | #793 | Zimbabwe | |
| P | camaldulensis | Red River Gum | New Zealand Tree | PO Box 435, |
| | | | Seeds | Rangiora, New |
| | | | | Zealand |
| Q | globulus | Tasmanian Blue | Same as above | Same as above |
| | | Gum/Blue | | |
| | | Gum | | |
| R | nitens | Shining Gum | Same as above | Same as above |
| S | saligna | Sydney Blue | Same as above | Same as above |
| | | Gum | | |

In vitro grown shoot cultures used to develop regeneration and transformation protocols were generated as follows. Seeds of 20 different seedlots of six different Eucalyptus species (see Table 1) were sterilized and germinated on ½ MS medium (Murashige & Skoog, Physiol. Plant. 15:473-497, 1962) using the following methods.

Protocol for Seed Sterilization and Germination

1. In a laminar flow hood under sterile condition, put seeds into 50 ml Falcon tube.
2. Wash with sterile MQ water once.
3. Add 70% ethanol for 5 minutes, and then remove 70% ethanol.
4. Fill the tubes with 15% bleach (commercial bleach, NaClO₃) and start the timer for 20 min. Place the tube in a shaker to keep mixing gently for the remainder of the 20 min.
5. Wash with sterile MQ water 3 times.
6. Plate on ½ MS medium.

Four weeks after germination, the shoots were sub-cultured on EMA4 or EEM medium (see Table 2 below) for multiplication and elongation. From each seedlot, shoot cultures were bulked up from 20 independent shoots separately. The shoot cultures from the same single shoot were treated as a clone. Shoot cultures were sub-cultured to fresh medium every four weeks.

TABLE 2
Media used in developing Eucalyptus internode transformation system

| Medium | | Cytokinins | Auxins | Agar/gelrite | Sucrose | Kanamycin | Timentin |
|---|---|---|---|---|---|---|---|
| code | Usage | (mg/l) | (mg/l) | (g/l) | (g/l) | (mg/l) | (mg/l) |
| EEM | MN | BA 0.01 | NAA | Agar 7 | 30 | | |
| | | | 0.01 | | | | |
| EMA4 | MN | BA 0.225 | | Agar 8 | 20 | | |
| EuCo12 | RE/CC | zeatin 2 | IAA | Agar 7 | 20 | | |
| | | | 0.2 | | | | |
| EuCo13 | RE/CC | zeatin 2.5 | IAA | Agar 7 | 20 | | |
| | | TDZ 0.01 | 0.2 | | | | |
| EuCo14 | RE/CC | zeatin 3 | IAA | Agar 7 | 20 | | |
| | | TDZ 0.01 | 0.2 | | | | |
| EuCo15 | RE/CC | zeatin 2.5 | IAA | Agar 7 | 20 | | |
| | | TDZ 0.1 | 0.2 | | | | |
| EuCo16 | RE/CC | zeatin 3 | IAA | Agar 7 | 20 | | |
| | | | 0.2 | | | | |
| EuCo17 | RE/CC | zeatin 4 | IAA | Agar 7 | 20 | | |
| | | | 0.2 | | | | |
| EuCo18 | RE/CC | zeatin 5 | IAA | Agar 7 | 20 | | |
| | | | 0.2 | | | | |
| EuCo19* | RE/CC | zeatin 3 | IAA | Gelrite 2.5 | 20 | *supplemented with | |
| | | TDZ 0.01 | 0.2 | | | 100 μM | |
| | | | | | | acetosyringone | |
| EuRt3 | RT/SL | BA 0.2 | NAA | Agar 7 | 20 | 50 | 250 |
| | | | 0.05 | | | | |
| EuSe1 | RE/SL | TDZ 1 | NAA | Agar 7 | 30 | 50 | 250 |
| | | | 0.1 | | | | |
| EuSe7 | RE/SL | zeatin 3 | IAA | Agar 7 | 20 | 30-50 | 250 |
| | | TDZ 0.01 | 0.2 | | | | |

The basal medium was MS salts and vitamins (Murashige & Skoog, Physiol. Plant. 15:473-497, 1962). For EuRt3, the basal medium is ½ MS salts. MN: maintenance, RE: regeneration, CC: co-cultivation, SE: selection, RT: rooting.

### EXAMPLE 2

### DEVELOPMENT OF A HIGHLY EFFICIENT REGENERATION SYSTEM

An efficient regeneration system is the prerequisite for development of a reliable plant transformation protocol. Plant genotype, explant type and age, and medium are key factors in determining the regeneration efficiency. A protocol to identify the best of these factors for Eucalyptus regeneration was developed as described below.

2.1 Determination of the Best Age of Shoot Internode Tissue for Adventitious Shoot Regeneration

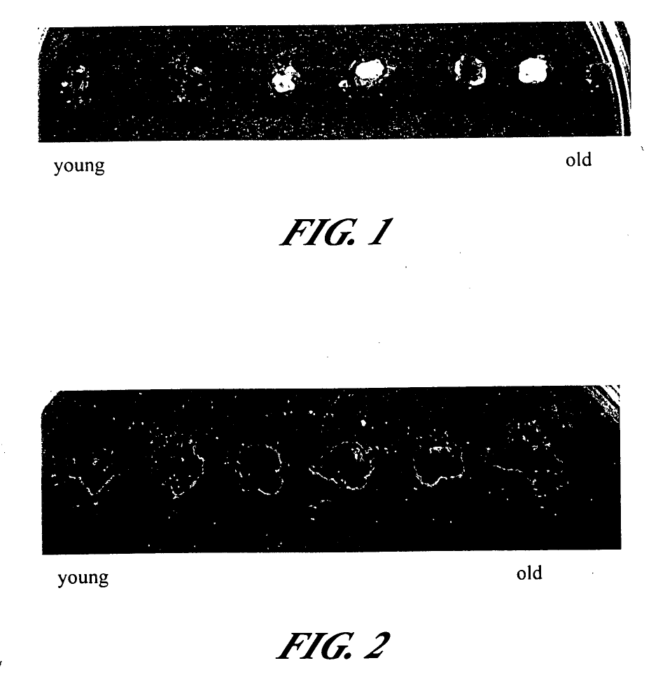

The regeneration of the Eucalyptus grandis clone A001 in medium EuCo14 was used to test the effects of internode age on adventitious shoot regeneration. A001 shoots were either continuously sub-cultured on the EEM or in the first subculture on the EMA4 from EEM. Shoot cultures at 8 to 10 days after sub-culturing are most suitable for providing internode tissue for the regeneration and transformation trials. An internode segment was collected from between two nodes from the second to 8^{th} node, counting from the top to the base of a shoot, by cutting the shoots using a scalpel and removing the node segments. The internode segments collected from the same shoot were cultured on a line according to their position order on the shoot. Segments from three shoots were cultured. Four weeks after culturing, the regeneration ability of each internode was visually assessed. The results showed that the younger internodes collected close to the top of the shoots had a much higher level of regeneration than the internode segments collected close to the base of the shoots. The younger the internode, the higher was the regeneration ability (see FIG. 1). In several subsequent experiments, only the top two internodes were used.

2.2 Internode Orientations

The regeneration medium EuCo14 and the Eucalyptus grandis clone 8 were used to test the effects of internode segment orientation on adventitious shoot regeneration. The internode explants were cultured in three different orientations: top-end-up; top-end-down; and sideways. As shown in Table 3, both the top-end-up and sideways orientations gave much better regeneration levels than the top-end-down orientation. The experiment was repeated with the clone D109 and a similar result was obtained. As it is time consuming to identify the top-end of internode segments, internode segments were cultured sideways, or horizontally, in subsequent experiments.

TABLE 3
The effect of internode orientation and maintenance medium on
shoot regeneration.

| | Previous | | Regeneration |
|---|---|---|---|
| Orientation | medium | No. explants | Rate |
| Top-end up | EMA4 | 14 | 43% |
| Top-end | EMA4 | 17 | 6% |
| down | | | |
| Sideways | EMA4 | 18 | 67% |
| Top-end up | EEM | 13 | 15% |
| Top-end | EEM | 12 | 0% |
| down | | | |
| Sideways | EEM | 12 | 0% |

2.3 Pretreatment of Shoot Cultures

The quality and physiological status of the shoot cultures that are used to provide the internode explants are likely to be important for the regeneration. Two types of shoot cultures produced on two different shoot maintenance media, EMA4 and EEM, were tested. As shown in Table 2, EMA4 contains a slightly higher level of the cytokinin benzyladenine (BA) than EEM. Shoots grown on EMA4 had shorter internode segments and more branches than shoots grown on EEM. The EMA4 shoots are likely to have a higher level of cell division activity than the EEM shoots, while EEM shoots may have a better cell elongation. Cell division activity in explant tissues is important to Agrobacterium-mediated transformation (Villemont et al., Planta 201:160-172, 1997).

Shoots of the E. grandis clone 8 from the EMA4 and EEM medium were used to provide internode segments for regeneration on medium EuCo14. The explants collected from the EMA4 medium gave a much higher level of regeneration than those collected from the EEM medium (Table 3). This experiment was repeated once. In subsequent experiments, shoot cultures grown on the EMA4 medium were used.

After the shoots were continuously sub-cultured on the EMA4 medium for more than 4 months, two trials were carried out to further investigate the effect of internodes age on regeneration using the Eucalyptus clone 8 and C101 respectively. Internode segments between the 2^{nd} and 8^{th} nodes from 10 different shoots for each clone were cultured on EuCo14 medium. Four weeks later, each internode was found to regenerate multiple shoots regardless their age (FIG. 2). This result is different from the previous result described in Example 2.1, where only young internodes produced shoots and old ones did not. The likely reason for this difference is that the quality of shoot cultures used to supply the internodes has been greatly improved after a number of subcultures on the EMA4 medium. These results suggest that more internodes can be used for regeneration when a high quality shoot culture is established. This improvement will reduce the limitation on explant supply and the variation in regeneration from different aged internodes.

2.3 Regeneration Medium

In previous studies using leaf primordia as explants, a wide range of medium was tested for regeneration from leaf primordia. It was determined that the cytokinin zeatin at a concentration of 2 mg/l was important for regeneration. Based on this result, seven media were tested with internode segments. These seven media, EuCo12-EuCo18, contain zeatin at different concentrations and in combination with another cytokinin thiadiazuron (TDZ) and auxin IAA (see Table 2 for medium details).

The E. grandis clone A001 was tested in four media, EuCo12-EuCo15. As shown in Table 4, EuCo14 gave the highest level of regeneration. In follow-up experiments with two different E. grandis clones (C101 and E. grandis 8), EuCo14 gave consistently good regeneration (Table 4). EuCo14 was thus used as the standard regeneration medium in subsequent experiments.

TABLE 4
The effect of medium on shoot regeneration.

| | | |
|---|---|---|
| | Number of | Regeneration |
| Medium | explants | rate |

Expt. I. Eucalyptus clone A001

| | | |
|---|---|---|
| EuCo12 | 14 | 7% |
| EuCo13 | 14 | 43% |
| EuCo14 | 16 | 56% |
| EuCo15 | 13 | 46% |

Expt. II. Eucalyptus clone C101

| | | |
|---|---|---|
| EuCo12 | 50 | 76% |
| EuCo13 | 50 | 72% |
| EuCo14 | 50 | 88% |
| EuCo15 | 50 | 92% |
| EuCo16 | 50 | 30% |
| EuCo17 | 50 | 44% |

Expt. III. Eucalyptus clone 8

| | | |
|---|---|---|
| EuCo12 | 50 | 96% |
| EuCo13 | 50 | 90% |
| EuCo14 | 50 | 96% |
| EuCo15 | 50 | 70% |
| EuCo16 | 50 | 94% |
| EuCo17 | 50 | 88% |

Following the tests described above, a preferred regeneration protocol was employed, which uses internodes collected from shoots continuously maintained on the EMA4 medium and cultures these internode explants sideways on the EuCo14 regeneration medium. Using the preferred protocol, multiple shoot regeneration from each internode segment culture has routinely been achieved (FIG. 3). The regenerated shoots can be elongated and rooted in the EuRt3 medium containing no kanamycin and developed into plants.

### EXAMPLE 3

### REGENERATION OF TRANSGENIC PLANTS

3.1 Preparation of Agrobacterium Cultures for Plant Transformation

To prepare Agrobacterium cultures for Eucalyptus transformation, 5 ml YEP (Yeast extract 10 g/l, peptone 10 g/l, NaCl 5 g/l) supplemented with 50 mg/l rifamycin and 50 mg/l kanamycin was inoculated with Agrobacterium containing a pART27-based binary vector. The cultures were placed in an incubator at 28° C. with vigorous shaking (200 rpm) overnight. In the early morning, 30 ml YEP containing 50 mg/l rifamycin and 50 mg/l kanamycin was inoculated with 3 ml of the overnight cultures, and placed in the same incubator for approximately 5 hours. In the afternoon, the Agrobacterium culture was removed from the incubator and its cell density was determined using a spectrophotometer by taking OD readings at 600 rm. The OD600 reading normally was around 1.0, indicating cell growth in its log-phase. The Agrobacterium cells were pelleted in a centrifuge at 5000 rpm for 10 minutes. The supernatant was discarded and cells were re-suspended in MS liquid medium to adjust the cell density at a particular OD600 reading, for example 0.8. The cells were stored on ice before they were used in the same day.

The pART27 vector contains a nptII gene in the T-DNA region for conferring kanamycin resistance (Gleave, Plant Mol. Biol. 20:1203-1207, 1992). All experiments to develop the inventive internode based Eucalyptus transformation protocol, employed pART69 derived from pART27 with an additional GUS gene in the T-DNA region (Ampomah-Dwamena et al., Plant Physiol. 130:605-617, 2002).

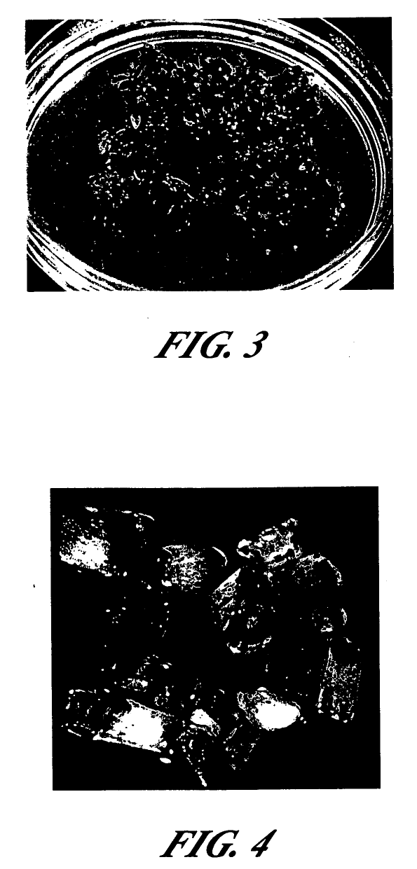

Although a number of Agrobacterium tumefaciens strains (e.g. AGL1, GV3101, EHA101, C58C1) can be used in the present protocol, use of the strain LBA4404, which is a non-hypervirulent strain and gives more single T-DNA copy insertions in plants, is preferred. It is believed that expression of a transgenic trait is more stable in plants containing single copy T-DNA than in plants containing multiple copies of T-DNA. For plants that are recalcitrant to Agrobacterium-mediated transformation, hypervirulent strains are normally chosen for improving the level of transformation. However, this often produces transgenic plants containing multiple copies of T-DNA and having a complex T-DNA integration pattern. Using the protocol described herein, a very high level of transformation can be achieved with the less virulent strain LBA4404. As shown in FIG. 4, strong transient GUS expression was observed on internode explants after inoculation with Agrobacterium LBA4404 containing the binary vector pART69.

3.2. Transformation of Eucalyptus Internode Explants with Agrobacterium Containing a Binary Plant Transformation Vector

Internode segment explants were prepared as described in Example 2 above. The explants were placed on a co-cultivation medium in a sideways orientation. The co-cultivation medium was usually EuCo19 (Table 2), although a similar medium could be employed. EuCo19 was based on the regeneration medium EuCo14, supplemented with 100 μM of acetosyringone. In addition, EuCo19 was solidified with gelrite while EuCo14 was solidified with agar. After 50 internode explants were placed on one medium plate, 1-2 μl of Agrobacterium cells were applied to each internode. The plate was then sealed and incubated at 22° C. under low intensity light (300 lux) for three days.

Both agar and gelrite were initially tested for the co-cultivation medium. It was found that, in comparison with the agar medium, the gelrite medium more readily absorbed the liquid Agrobacterium cultures applied to the internodes. The explants also appeared to be healthier after the co-cultivation on gelrite medium than on agar medium. Gelrite, rather than agar, was therefore used for the co-cultivation medium.

After 3 days of co-cultivation, the explants were transferred to regeneration/selection medium containing 20-50 mg/l kanamycin and 250 mg/l timentin. In a number of experiments, 10-30 explants from each treatment were taken from the selection medium at day 4 on the medium and tested for transient transgene expression using a GUS staining procedure described in Example 3.6 below.

3.3. Determination of a Suitable Kanamycin Concentration for Internode Explants

In studies with leaf primordia, it was found that 50 mg/l kanamycin was suitable for selection of transgenic plants. The internode selection experiments were therefore started with this concentration of kanamycin, however it was found that this concentration is too high for internode explants. A range of kanamycin concentrations (0, 5, 10, 15, 20, 30, 40 and 50 mg/l) were then tested in four experiments to determine the minimum level of kanamycin for inhibiting regeneration from the wild type internode explants (Table 5).

Fifty internode explants of the E. grandis clone 8 or C101 were cultured on each of the eight media consisting of EuCo14 supplemented with kanamycin at eight different concentrations, namely 0, 5, 10, 15, 20, 30, 40 and 50 mg/l. As shown in Table 5, 86% to 100% regeneration was observed on medium with 0 mg/l kanamycin, 2 to 10% regeneration on medium with 5 to 20 mg/l kanamycin and no regeneration on medium with 30 to 50 mg/l kanamycin. As 30 mg/l kanamycin can completely inhibit the regeneration from wild type internode tissues, a high level of kanamycin (50 mg/1) may not be required for internode transformation even though it is usually used for leaf primordium transformation protocols. An excessive level of kanamycin kills non-transformed cells in the explant early in the selection stages. This will reduce the regeneration from transformed cells which would be surrounded by these dead non-transformed cells. The kanamycin concentration should be in the range that inhibits cell division and regeneration from non-transformed cells but does not kill these cells too rapidly.

TABLE 5
The effect of kanamycin level on shoot regeneration from
wild type explants

| | | |
|---|---|---|
| Kanamycin | No. | Regeneration Rate |

| level (mg/L) | Explants | Exp 1 | Exp 2 | Exp 3 | Exp 4 |
|---|---|---|---|---|---|
| 0 | 50 | 100% | 92% | 86% | 96% |
| 5 | 50 | nt | nt | 2% | 10% |
| 10 | 50 | nt | nt | 0% | 0% |
| 15 | 50 | nt | nt | 0% | 0% |
| 20 | 50 | 0% | 4% | nt | nt |
| 30 | 50 | 0% | 0% | nt | nt |
| 40 | 50 | 0% | 0% | nt | nt |
| 50 | 50 | 0% | 0% | nt | nt |

In a follow-up experiment, 425 internode explants of the E. grandis clone C101 were co-cultivated with LBA4404 containing the binary vector pART69 and then transferred to selection media containing five different concentrations of kanamycin. The regeneration efficiencies were 100%, 17%, 8%, 3% and 1% from kanamycin concentrations of 0, 20, 30, 40 and 50 mg/l, respectively. A similar result was achieved for the clone Eg 8 in an independent experiment (Table 6).

TABLE 6
The effect of kanamycin level on shoot regeneration from
co-cultivated explants

| | | |
|---|---|---|
| Kanamycin | No. | Regeneration |
| level (mg/l) | explants | rate |

Exp I, Eucalyptus clone C101

| | | |
|---|---|---|
| 0 | 25 | 100% |
| 20 | 100 | 17% |
| 30 | 100 | 8% |
| 40 | 100 | 3% |
| 50 | 100 | 1% |

Exp II, Eucalyptus clone Eg8

| | | |
|---|---|---|
| 20 | 100 | 29% |
| 30 | 100 | 12% |
| 40 | 100 | 6% |
| 50 | 25 | 4% |

It is possible that there could be some non-transgenic plants regenerated from co-cultivated explants on selection medium with 20 mg/l and 30 mg/l kanamycin. Non-transgenic plants (escapes) can normally be regenerated from co-cultivated explants on a lower level of kanamycin, although no plants can be regenerated from non-co-cultivated explants on the same kanamycin kevel. Regeneration from transiently transformed cells can partially account for the escapes. Taking all these factors into account, the preferred protocol is to culture the co-cultivated explants on 30 mg/l kanamycin for 4 weeks and then transfer them to 50 mg/l kanamycin.

3.4. Testing of Agrobacterium density

To determine a suitable density of Agrobacterium cells for inoculation of Eucalyptus internode explants, LBA4404 cells containing the pART69 vector were used at the densities of OD₆₀₀=0.1 and 0.8. The high density gave a much higher level of transient GUS expression (Table 6), indicating a high level of gene transfer from Agrobacterium to plant cells. In a follow-up experiment, OD₆₀₀=0.1, 0.4 and 0.8 were used. OD₆₀₀=0.8 gave the highest level of transient GUS expression (Table 7). An Agrobacterium density of OD₆₀₀=0.8 was thus chosen as the preferred density for subsequent experiments.

TABLE 7
The effects of Agrobacterium cell density on transient GUS expression

| | | | |
|---|---|---|---|
| | | | No. GUS |
| Agrobacterium | Number | Gus positive | Foci per |
| OD | explants | explants (%) | explant |

Experiment I

| | | | |
|---|---|---|---|
| MS control | 25 | 0% | 0 |
| OD₆₀₀ = 0.1 | 175 | 67% | 1 |
| OD₆₀₀ = 0.8 | 175 | 100% | 10 |

Experiment II

| | | | |
|---|---|---|---|
| OD₆₀₀ = 0.1 | 100 | 90% | 2 |
| OD₆₀₀ = 0.4 | 100 | 100% | 10 |
| OD₆₀₀ = 0.8 | 75 | 100% | 11 |

3.5. Regeneration of Transgenic Plants

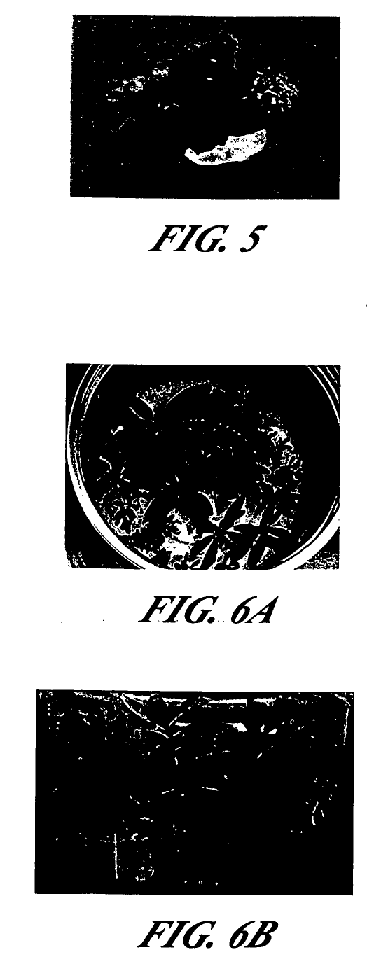

To regenerate transgenic plants, internode explants were transferred to selection medium after three days of co-cultivation. The selection medium was normally EuSe7, which was derived from the regeneration medium EuCo14 by supplementing with 250 mg/l timentin and 30 mg/l kanamycin (Table 2). After four weeks on this medium, regeneration of shoots was visible on some of the explants (FIG. 5). The explants plus regenerating shoots were transferred to fresh selection medium containing 50 mg/l kanamycin for four weeks. At the end of this culture, putative transgenic shoots became large enough for separation from the explants and were transferred to the EuRt3 medium containing 250 mg/l timentin and 50 mg/l kanamycin for shoot elongation and multiplication. The elongated shoots (2-3 cm long) were transferred to the rooting medium EuRt3 (Table 2) for root induction (FIG. 6). Two-three weeks later, rooted plantlets were transplanted into soil.

3.6. Analyses of Transgenic Plants

GUS Staining

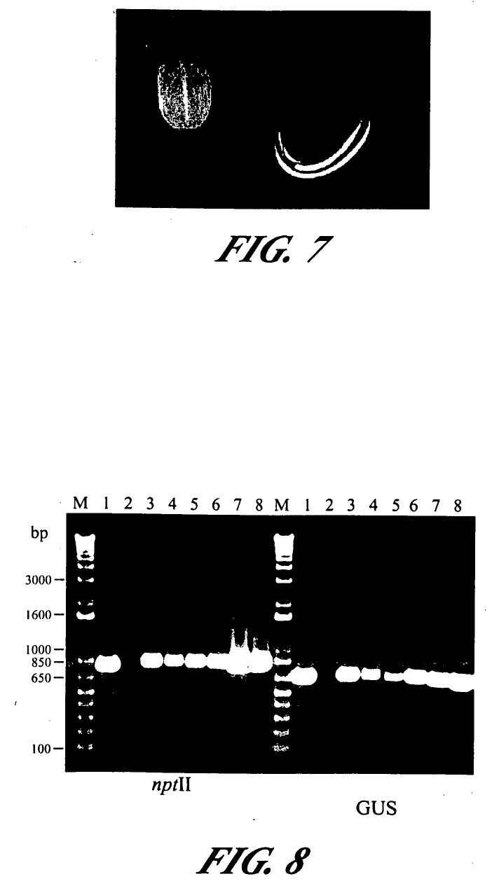

For detection of transient GUS expression, intemode explants were used at 6 days post inoculation with Agrobacterium. For detection of stable GUS expression, young leaf tissue from putative transgenic plants was used. The leaf tissue was collected from kanamycin resistant plants grown on medium containing kanamycin for more than 8 weeks, or from soil grown plants. The kanamycin-resistant transgenic plants containing the pART69 vector were GUS positive while wild type Eucalyptus plants were GUS negative (FIG. 7). The GUS staining protocol was as follows.

GUS staining protocol:

1. The GUS histochemical staining solution was prepared as described by Jefferson (Plant Mol. Biol. Rep. 5:387-405, 1987).
2. Add GUS staining solution into the wells of a multi-well plate.
3. Put Eucalyptus internode explants or leaf tissue into the wells with GUS staining solution.
4. Vacuum 2 times, 5 minutes each time at 35° C.
5. Place the multi-well plate in 28° C. incubator overnight.
6. Remove GUS staining solution and add 70% ethanol to extract chlorophyll. Blue GUS staining was recorded by photographing, and the number of explants with GUS staining and number of GUS staining foci per explant were counted.
   PCR

To confirm the presence of the T-DNA constructs in the transformed Eucalyptus plants, PCR was performed with Expand High Fidelity PCR System (Roche Diagnostics). Genomic DNA was isolated from Eucalyptus young leaf tissues as described by Doyle and Doyle (Focus 12:13-15, 1990). Two primers were designed and used to amplify an 804 bp DNA fragment from the nptII gene. Similarly, two primers were designed and used to amplify a 677 bp fragment from the GUS gene. PCR conditions were as follows: initial denaturation at 95° C. for 2 min, 25 cycles of 95° C. for 30 s, 58° C. for 1 min, and 72° C. for 1 min plus a final extension at 72° C. for 5 min.

DNA fragments of the nptII and GUS gene were amplified with expected size from the six transgenic plants tested but not from a non-transgenic plant (FIG. 8). This result confirms the transgenic status of the Eucalyptus plants produced with the transformation protocols described above.

Southern Analysis

Genomic DNA was isolated from Eucalyptus young leaf tissues as described by Doyle and Doyle (Focus 12:13-15, 1990). DNA (20 μg) was digested with BamHI, and EcoRV in separate reactions. The digests were separated on 1% (w/v) agarose gel and transferred onto Hybond N⁺ membrane (Amersham, Buckinghamshire, UK). A 1.7-kb fragment from the left border region and the nptII gene was labeled and used as a probe. Hybridization and washing of blots were as described previously (Church and Gilbert, Proc. Natl. Acad. Sci. USA 81:1991-1995, 1984). Hybridization signals were visualized with the Storm 840 Phospho-Imaging system (Alphatech, Arlington, Va.) and ImageQuant software (Molecular Dynamics, Sunnyvale, Calif.).

One to three hybridization bands were detected from each of the six independent transgenic plants tested. The hybridization bands were generally larger than 5 kb and of variable size. The Southern analysis was designed to detect the T-DNA left border and plant DNA junctions in transgenic plants. The presence of high molecular weight bands of variable size is strong evidence for integration of T-DNA into the plant genome. One to three bands were detected from the different transgenic plants, indicating one to three T-DNA insertions. No hybridization bands were detected from DNA isolated from the non-transgenic control plants.

### EXAMPLE 4

### PREFERRED PROTOCOLS

A preferred transformation and regeneration protocol, based on the previous Examples and the disclosure made herein, is as follows.

4.1. Preparation of in vitro Shoot Cultures to provide Internode Explants

Subculture shoots on the EMA4 medium every 4 weeks.

4.2. Preparation of Agrobacterium Cultures for Transformation

Prepare Agrobacterium LBA4404 cell cultures by an overnight culture, followed with a 5 hour culture, adjust cell density with MS liquid medium to OD₆₀₀=0.8. Store the cells on ice for use on the same day.

4.3. Preparation of Internodes for Transformation

Collect the internode segments between node 2 and 3, or 3 and 4, at ten days after the shoots are subcultured to fresh medium. Place the internode segments in a sideways orientation on the co-cultivation medium EuCo19. When the shoot cultures are of high quality, more internodes (up to 6) from a shoot can be collected.

4.4. Inoculation of Internodes with Agrobacterium

Apply a 2 μl drop of Agrobacterium cells to each internode explant. Co-cultivate for three day under low intensity light at 22° C.

4.5. Regeneration of Transgenic Shoots

Transfer co-cultivated explants to the selection medium EuSe7 containing 30 mg/l kanamycin for 4 weeks, then transfer to EuSe7 containing 50 mg/l kanamycin for 4 weeks. Transfer putative kanamycin resistant shoots to the EuRT3 medium containing 250 mg/l timentin and 50 mg/l kanamycin for 4 weeks for shoot elongation.

4.6. Rooting of Transgenic Shoots and establishing Transgenic Plants in Soil

Transfer elongated shoots to the rooting medium EuRt3 for 2-3 weeks. Transplant rooted plants into soil.

All references cited herein, including patent references and non-patent publications, are hereby incorporated by reference in their entireties.

While in the foregoing specification this invention has been described in relation to certain preferred embodiments, and many details have been set forth for purposes of illustration, it will be apparent to those skilled in the art that the invention is susceptible to additional embodiments and that certain of the details described herein may be varied considerably without departing from the basic principles of the invention.

1. A method for producing genetically modified plants of a Eucalyptus species, comprising:
(a) culturing shoots of a target plant selected from the Eucalyptus species and collecting internode stem segments from the shoots;
(b) transforming a culture of a non-hypervirulent Agrobacterium strain with a genetic construct comprising a polynucleotide of interest and a selection marker that confers resistance to a selection agent;
(c) transforming the internode stem segments by incubating the segments with the transformed Agrobacterium culture;
(d) cultivating the internode stem segments in a first medium comprising a first concentration of the selection agent;
(e) cultivating internode stem segments that survive exposure to the first medium in a second medium comprising a second, higher, concentration of the selection agent and identifying transformed shoots containing the polynucleotide of interest; and
(d) regenerating transformed plants from the transformed shoots.

2. The method of claim 1, wherein the plant is of the Eucalyptus grandis species.

3. The method of claim 1, wherein the Agrobacterium strain is Agrobacterium tumefaciens strain LBA4404.

4. The method of claim 1, wherein the internode stem segments are taken from the top of the shoots.

5. The method of claim 1, wherein the selection agent is kanamycin.

6. The method of claim 1, wherein the genetic construct comprises genetic material that is homologous to the genome of the target plant.

7. The method of claim 1, wherein the genetic construct comprises genetic material that is heterologous to the genome of the target plant.

8. The method of claim 1, wherein the genetic construct comprises genetic material that affects one of the following phenotypic properties of the target plant: insect tolerance; disease resistance; herbicide tolerance; sterility; rooting ability; temperature tolerance; drought tolerance; salinity tolerance; wood properties; and growth rate.

9. The method of claim 1, wherein the genetic construct comprises genetic material encoding a polypeptide of interest or a functional portion of a polypeptide of interest.

10. The method of claim 1, wherein the genetic construct comprises an antisense copy of a gene or a portion of a gene encoding a polypeptide of interest or a functional portion of a polypeptide of interest.

11. A genetically modified plant produced according to the method of claim 1.

12. A plant material or plant derived from the genetically modified plant of claim 11.

13. A plant product derived from the genetically modified plant of claim 11.
